SEQUENCE LISTING (4 sequences)

SEQ ID NO: 1 - protein, 34 aa, Xanthomonas sp.
LTPEQVVAIASHDGGKQALETVQRLLPVLCQAHG

SEQ ID NO: 2 - DNA, 23 bp, Artificial Sequence
  source: /note=“Description of Artificial Sequence Synthetic oligonucleotide”
ccatcatatgcataccgaaatgc

SEQ ID NO: 3 - DNA, 23 bp, Artificial Sequence
  source: /note=“Description of Artificial Sequence Synthetic oligonucleotide”
  modified_base (21)..(21): a, c, t, g, unknown or other
gcatttcggtatgcatatgangg

SEQ ID NO: 4 - RNA, 104 nt, Artificial Sequence
  source: /note=“Description of Artificial Sequence Synthetic polynucleotide”
gcauuucgguaugcauaugaaugaguuuuagagcuagaaauagcaaguuaaaauaaggcuaguccguuaucaacuugaaaaaguggcaccgagucggugcuuuu

(19) United States
(12) Patent Application Publication
(10) Pub. No.: US 2018/0327738 A1
(43) Pub. Date: Nov. 15, 2018

(54) STABILIZED REAGENTS FOR GENOME MODIFICATION

(71) Applicant: LIFE TECHNOLOGIES CORPORATION, Carlsbad, CA (US)

(72) Inventors: Namritha RAVINDER, San Diego, CA (US); Robert Jason POTTER, San Marcos, CA (US); Jason CARTE, Carlsbad, CA (US)

(21) Appl. No.: 15/777,182

(22) PCT Filed: Nov. 18, 2016
(86) PCT No.: PCT/US2016/062844

§ 371 Date: May 17, 2018

Related U.S. Application Data
(60) Provisional application No. 62/257,951, filed on Nov. 20, 2015.

(51) Int. Cl. C12N 15/10; C12N 9/22

(52) U.S. Cl. CPC C12N 15/1003; C07K 2319/81; C12N 9/22

(57) ABSTRACT

The present disclosure generally relates to compositions and methods for genetic modification of cells. In particular, the disclosure relates to stabilized reagents for genome alteration (e.g., site specific nucleases), as well as stabilized reagents for non-genetic code altering modification (e.g., DNA methylation). Stabilization methods include storage of reagents at suitable temperatures, conversion to dry forms, and chemical modifications.

### FIELD

The present disclosure generally relates to compositions and methods for genetic modification of cells. In particular, the disclosure relates to stabilized reagents for genome alteration (e.g., site specific nucleases), as well as stabilized reagents for non-genetic code altering modification (e.g., DNA methylation). Stabilization methods include storage of reagents at suitable temperatures, conversion to dry forms, and chemical modifications.

### BACKGROUND

A number of genome-interacting systems, such as designer zinc fingers, transcription activator-like effectors (TALs), CRISPRs, and homing meganucleases, have been developed. One issue with these systems is that they require a both the identification of target sites for alteration and the designing of a reagents specific for those sites, which is often laborious and time consuming. In one aspect, the invention allows for the efficient design, preparation, and use of genome interaction reagents.

### SUMMARY

The present disclosure relates, in part, to compositions and methods for modification of nucleic acid molecules. There exists a substantial need for efficient systems and techniques for modifying genomes. This invention addresses this need and provides related advantages.

In some aspects, the invention includes method for preparing one or more (e.g., one, two, three, four, five, six, etc.) stabilized gene altering reagents, as well as stabilized gene altering reagents prepared by such methods. In some instances, these methods comprise (a) preparing one or more gene altering reagent in a solvent, and (b) removing more than 80% of the solvent of (a). Further, the solvent may be aqueous, organic (e.g., one or more alcohol), or a mixture of an aqueous solvent and one or more organic solvent. The solvent may be removed by any number of means, including lyophilization, spray drying, spray freeze drying, supercritical fluid drying, or vacuum centrifugation. In some instances, between 80% and 99.5% (e.g., from about 80% to about 95%, from about 80% to about 90%, from about 85% to about 95%, from about 85% to about 99%, from about 90% to about 99%, from about 90% to about 98%, from about 90% to about 97%, from about 90% to about 96%, from about 93% to about 99%, etc.) of the solvent may be removed from the one or more gene altering reagents.

In some instances, at least one of the one or more gene altering reagents may be one or more reagents selected from the group consisting of (a) a TAL effector-nuclease fusion protein, (b) a nucleic acid molecule encoding a TAL effector-nuclease fusion protein, (c) a zinc finger-nuclease fusion protein, (d) a nucleic acid molecule encoding a zinc finger-nuclease fusion protein, (e) a Cas9 protein, (f) a nucleic acid molecule encoding a Cas9 protein, (g) a guide RNA, and (h) a nucleic acid molecule encoding a guide RNA.

In some instances, individual gene altering reagents are placed in two or more wells of a multiwell plate. Further, individual gene altering reagents may be added to wells of the multiwell plate while solubilized in a solvent. Also, some or all of the solvent may be removed from the individual gene altering reagents while the individual gene altering reagents are in wells of the multiwall plate.

One or more donor nucleic acid molecule (e.g., donor DNA) may be co-mixed with the gene altering reagents. For example, cells may be prepared where different inserts are introduced into a specific chromosomal locus. Thus, the invention includes “libraries” of cells in which different nucleic acid segments are introduced at a specific locus. In some instances, the number of donor nucleic acid molecules may be from about 2 to about 10,000 (e.g., from about 5 to about 10,000, from about 20 to about 10,000, from about 50 to about 10,000, from about 90 to about 10,000, from about 200 to about 10,000, from about 400 to about 10,000, from about 800 to about 10,000, from about 2,000 to about 10,000, from about 2 to about 10,000, from about 100 to about 1,000, from about 200 to about 3,000, from about 150 to about 1,500, etc.).

The number of gene altering reagents present in a collection may vary greatly and may be from about 2 to about 10,000 (e.g., from about 5 to about 10,000, from about 20 to about 10,000, from about 50 to about 10,000, from about 90 to about 10,000, from about 200 to about 10,000, from about 400 to about 10,000, from about 800 to about 10,000, from about 2,000 to about 10,000, from about 2 to about 10,000, from about 100 to about 1,000, from about 200 to about 3,000, from about 150 to about 1,500, etc.). Such gene altering reagents may be placed in different wells of one or more multiwell plates. In many instances, such individual gene altering reagents will bind to different nucleotide sequences of the genome of the same organism.

In some instances, solvents (e.g., aqueous solutions) in contact with gene alerting reagents may contain one or more component selected from the group consisting of (a) one or more buffer, (b) one or more protease inhibitor, (c) one or more nuclease inhibitor, (d) one or more salt, (e) one or more carbohydrate, (f) one or more transfection reagent, (g) one or more polyamine, and (h) one or more culture medium. In specific instances, the carbohydrate is one or more of the following: sucrose, trehalose, lactosucrose, or a cyclodextrin.

Further, the pH of solvents (e.g., aqueous solutions) prior to the removal of the water may be between 4 to 11 (e.g., from about 4 to about 7, from about 4 to about 6.5, from about 4 to about 8, from about 6.5 to about 11, from about 6.5 to about 10, from about 7 to about 11, from about 7 to about 10, etc.).

The invention further includes methods for storing one or more gene altering reagents, as well as stored gene altering reagents prepared by such methods. In some instances, these methods comprise (a) preparing one or more gene altering reagents in aqueous solution, (b) removing more than 90% of the water from the aqueous solution prepared in (a), and (c) placing one or more gene altering reagents under conditions where greater than 75% of gene altering functional activity is retained after 30 days of storage. In specific instances, greater than 75% (e.g., at least 80%, at least 85%, at least 90%, at least 95%, etc.) of gene altering functional activity is retained after at least 30 (e.g., at least 30, at least 60, at least 90, at least 120, etc.) days of storage.

Further, more than one of the one or more gene altering reagents may be stored in the same storage container (e.g., a multiwell plate). The individual stored gene altering reagents may bind to different nucleotide sequences of the genome of the same organism. Additionally, the one or more gene altering reagents may be stored at −70° C., −20° C., 4° C., or between 20° C. and 30° C. (e.g., from about −70° C. to about 30° C., from about −22° C. to about 30° C., from about 2° C. to about 30° C., from about −4° C. to about 30° C., from about −10° C. to about 30° C., from about −15° C. to about 30° C., from about −22° C. to about 10° C., etc.).

The invention further includes compositions comprising one or more stabilized gene altering reagents. Such compositions comprise one or more gene altering reagent, wherein the moisture content of the gene altering reagent is less than 10% (w/w) (e.g., from about 0.3% to about 7%, from about 0.5% to about 8%, from about 0.5% to about 5%, from about 0.2% to about 4%, from about 0.2% to about 3%, etc.).

Further, compositions of the invention may comprise at least one of the one or more gene altering reagents selected from the groups consisting of (a) a TAL effector-nuclease fusion protein, (b) a nucleic acid molecule encoding a TAL effector-nuclease fusion protein, (c) a zinc finger-nuclease fusion protein, (d) a nucleic acid molecule encoding a zinc finger-nuclease fusion protein, (e) a Cas9 protein, (f) a nucleic acid molecule encoding a Cas9 protein, (g) a guide RNA, and (h) a nucleic acid molecule encoding a guide RNA. As noted above, one or more donor nucleic acid molecule (e.g., donor DNA) may be co-mixed with the gene altering reagents.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a representative diagram of some aspects of the invention. This diagram shows examples of reagents for single component (e.g., zinc finger and TAL systems) and multi-component gene altering systems (e.g., CRISPR systems, such as Cas9 and Cpf1 systems). Reagents for the representative single components systems may be DNA, mRNA, and protein. Any one or more of these may be introduced into cells for genome alteration. Reagents for the representative multi-components systems may be DNA, RNA, and/or protein. One or both of these may be introduced into cells for genome alteration. DNA and mRNA reagents enter the cells as precursors that are them converted into functional RNA (e.g., gRNA) or proteins (e.g., Cas9, Cpf1, zinc finger nuclease or Tal nuclease).

FIG. 2 shows an exemplary plate format for use in one aspect of the invention. The plate contains a 6 by 8 array of wells where each well is identified by a number and letter combination. Wells A,1 and A,6 contain no gene altering reagents and thus are control wells.

FIG. 3 is a schematic drawing of the modular structure of a representative naturally occurring TAL protein. This protein is composed of an amino terminal end (N), a central array comprising a variable number of 34-amino acid repeats indicated by ovals with hypervariable residues at positions 12 and 13 that determine base preference, and a carboxyl terminal end (C) comprising a nuclear localization signal (NLS) and a transcription activator (AD) domain.

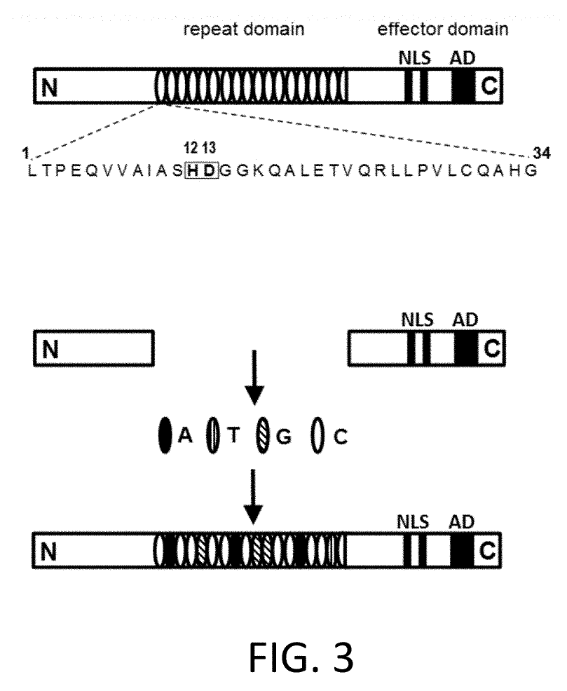

FIG. 4 is a schematic of a guide RNA molecule (104 nucleotides) showing the guide RNA bound to both Cas9 protein and a target genomic locus. Hairpin Region 1 is formed by the hybridization of complementary crRNA and tracrRNA regions joined by the nucleotides GAAA. Hairpin Region 2 is formed by a complementary region in the 3′ portion of the tracrRNA.

FIG. 5 shows a workflow for synthesizing guide RNA using DNA oligo templates. Guide RNA encoding DNA template is generated using assembly PCR. Components of this assembly reaction include 1) a target specific DNA oligo (encodes the crRNA region), 2) DNA oligo specific to the bacterial promoter used for in vitro transcription (in this case T7 promoter), and 3) overlapping PCR products encoding tracrRNA region. A fill in reaction followed by PCR amplification is performed in a Thermo cycler using DNA polymerase enzyme (in this case high fidelity PHUSION® Taq DNA polymerase) to generate full length gRNA encoding templates. Following PCR assembly the resulting DNA template is transcribed at 37° C. to generate target specific gRNA using in vitro transcription reagents for non-coding RNA synthesis (in this case MEGASHORTSCRIPT™ T7 kit). Following synthesis the resulting gRNA is purified using a column or magnetic bead based method. Purified in vitro transcribed guide RNA is ready for co-transfection with Cas9 protein or mRNA delivery in a host system or cell line of interest.

FIG. 6 is a schematic showing a nicking based nucleic acid cleavage strategy using a CRISPR system. In the top portion of the figure, two lines represent double-stranded nucleic acid. Two nick sites are indicated by Site 1 and Site 2. These sites are located within a solid or dashed box indicating the region of the nucleic acid that interacts with the CRISPR/Cas9 complex. The lower portion of the figure show nicking actions that result in two closely positioned nicks in both strands.

FIG. 7 shows the cleavage efficiency of IVT guide RNAs in U2OS-Cas9 cell line upon reconstitution of drying and lyophilization. No guide RNA was in the wells labeled as CPFS1 T2.

### DETAILED DESCRIPTION

### Definitions

As used herein the term “nucleic acid alterations” refers to alteration or changes to genetic code or non-code based nucleic acid modifications. Genetic code alteration refers nucleotide sequence changes of nucleic acid molecules. Non-code based nucleic acid alteration refers to nucleic acid modifications, such as methylation, that do not involve nucleotide sequence alterations, as well as modifications that result in alteration of gene expression (e.g., histone acetylation, promoter activation, promoter repression, etc.). Thus, a functional TAL-VP16 fusion protein would result in non-code based nucleic acid alteration when involved in the transcription of DNA.

As used herein the term “gene altering reagent” refers a composition that has one or more nucleic acid alteration activity or contains a component of a complex that has one or more nucleic acid alteration activity. Exemplary gene altering reagents are reagents that contain functional zinc finger-FokI fusion proteins, functional TAL-VP16 fusion protein, and gRNA molecules that are capable of directing a Cas9 protein a specific nucleotide region of a target nucleic acid molecule.

As used herein the term “stabilized gene altering reagent” refers a reagent that may be stored for a period of time with minimal loss of functional activity. Parameters related to this definition are set out herein.

As used herein the term “CRISPR system” refers to a collection of CRISPR proteins and nucleic acid that, when combined, result in at least CRISPR associated activity (e.g., the target locus specific, double-stranded cleavage of double-stranded DNA).

As used herein the term “CRISPR complex” refers to the CRISPR proteins and nucleic acid (e.g., RNA) that associate with each other to form an aggregate that has functional activity. An example of a CRISPR complex is a wild-type Cas9 (sometimes referred to as Csn1) protein that is bound to a guide RNA specific for a target locus.

As used herein the term “CRISPR protein” refers to a protein comprising a nucleic acid (e.g., RNA) binding domain nucleic acid and an effector domain (e.g., Cas9, such as Streptococcus pyogenes Cas9). The nucleic acid binding domains interact with a first nucleic acid molecule either having a region capable of hybridizing to a desired target nucleic acid (e.g., a guide RNA) or allows for the association with a second nucleic acid having a region capable of hybridizing to the desired target nucleic acid (e.g., a crRNA). CRISPR proteins can also comprise nuclease domains (i.e., DNase or RNase domains), additional DNA binding domains, helicase domains, protein-protein interaction domains, dimerization domains, as well as other domains.

CRISPR protein also refers to proteins that form a complex that binds the first nucleic acid molecule referred to above. Thus, one CRISPR protein may bind to, for example, a guide RNA and another protein may have endonuclease activity. These are all considered to be CRISPR proteins because they function as part of a complex that performs the same functions as a single protein such as Cas9.

In many instances, CRISPR proteins will contain nuclear localization signals (NLS) that allow them to be transported to the nucleus.

As used herein, the term “transcriptional regulatory sequence” refers to a functional stretch of nucleotides contained on a nucleic acid molecule, in any configuration or geometry, that act to regulate the transcription of (1) one or more structural genes (e.g., two, three, four, five, seven, ten, etc.) into messenger RNA or (2) one or more genes into untranslated RNA. Examples of transcriptional regulatory sequences include, but are not limited to, promoters, enhancers, repressors, and the like.

As used herein, the term “promoter” is an example of a transcriptional regulatory sequence, and is specifically a nucleic acid generally described as the 5′ region of a gene located proximal to the start codon or nucleic acid which encodes untranslated RNA. The transcription of an adjacent nucleic acid segment is initiated at the promoter region. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

As used herein, the terms “vector” refers to a nucleic acid molecule (e.g., DNA) that provides a useful biological or biochemical property to an insert. Examples include plasmids, phages, autonomously replicating sequences (ARS), centromeres, and other sequences which are able to replicate or be replicated in vitro or in a host cell, or to convey a desired nucleic acid segment to a desired location within a host cell. A vector can have one or more restriction endonuclease recognition sites (e.g., two, three, four, five, seven, ten, etc.) at which the sequences can be cut in a determinable fashion without loss of an essential biological function of the vector, and into which a nucleic acid fragment can be spliced in order to bring about its replication and cloning.

As used herein the term “nucleic acid targeting capability” refers to the ability of a molecule or a complex of molecule to recognize and/or associate with nucleic acid on a sequence specific basis.

As used herein the term “target locus” refers to a site within a nucleic acid molecule for gene altering reagent interaction (e.g., binding and cleavage). When the gene altering reagent is designed to cleave double-stranded nucleic acid, then the target locus is the cut site and the surrounding region recognized by the CRISPR complex. When the gene altering reagent is designed to nick double-stranded nucleic acid in close proximity to create a double-stranded break, then the region surrounding and including the break point is referred to as the target locus.

### Overview

The invention relates, in part, to compositions and methods for the genome alteration. In particular, the invention relates to stabilized reagents and methods for producing and using such reagents. Stabilization may result from storage conditions (e.g., temperature, humidity, etc.) or from chemical characteristics of reagents (e.g., chemically modified nucleotides, buffers, presence of reducing agents, etc.) being stored.

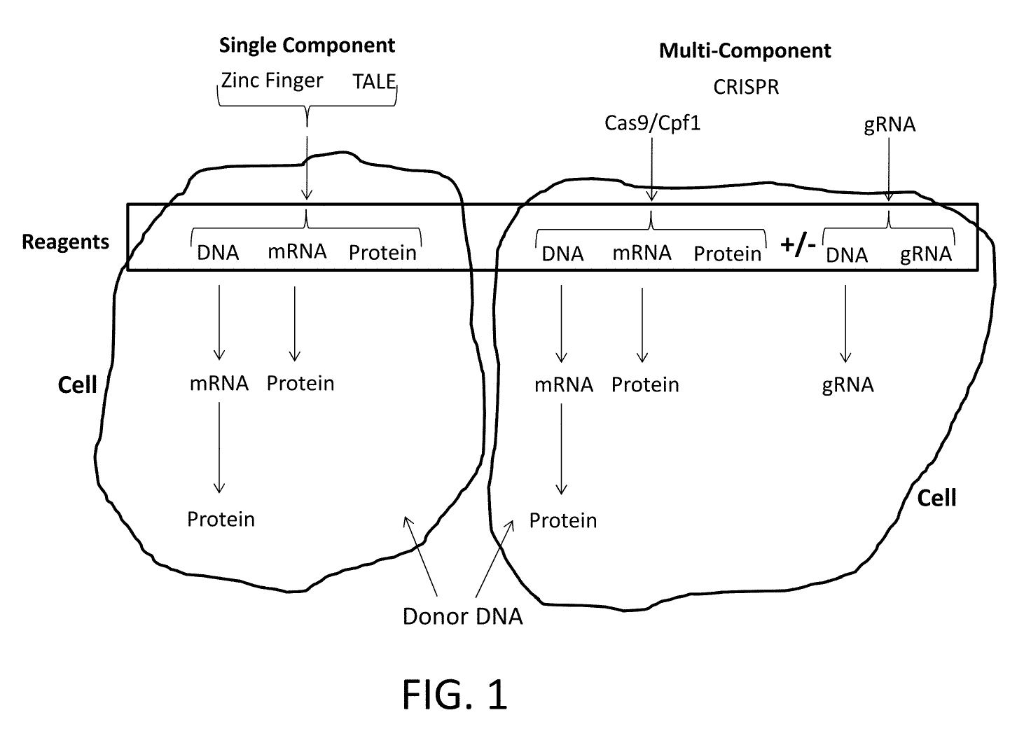

Using the schematic representation set out in FIG. 1 for purposes of illustration, two broad categories of gene altering reagents may be prepared: Single component and multi-component. Single component gene altering reagents refer to reagents that either are a gene alteration functional component or encode a gene alteration functional component. Thus, single component systems will typically comprise DNA, RNA, or protein. When the reagent is DNA, this DNA will typically be introduced into cells, where it is transcribed to form mRNA. The mRNA is then translated to generate protein as a gene alteration functional component (e.g., a zinc finger protein or a TAL protein).

Multi-component systems require more than one component for gene alteration activity. One example of this type of system is Cas9 based CRISPR systems. Systems such as this require a protein component (e.g., a Cas9 protein) and at least one nucleic acid component (e.g., a gRNA) for gene alteration activity. The protein component may be introduced into cells as a protein or encoded by mRNA or DNA that are introduced into the cell. Further, gRNA or DNA encoding gRNA may be introduced into cells that express one or more protein components of a multi-component system.

In most instances, the goal will be to either introduce into cells (1) one or more functional gene editing reagents (2) one or more nucleic acid molecule encoding gene editing reagents, or (3) a combination of one or more gene altering reagents that are ready to form gene altering complexes and one or more nucleic acid molecule encoding additional gene altering reagents.

In particular, the invention relates to combinations of proteins and nucleic acid molecules designed to interact with other nucleic acid molecules. In some instances, the invention relates to protein/nucleic acid complexes, where the nucleic acid component has sequence complementarity to a target nucleic acid molecule. In these systems, sequence complementarity between the complexed nucleic acid and the target nucleic acid molecule is the used to bring the complex into association with the target nucleic acid. Once this occurs, functional activities associated with the complex may be used to modify the target nucleic acid molecule.

### Non-Chemical Stabilization:

Non-chemical stabilization refers to stabilization means that do not involve chemical modification of functional components of gene altering reagents (e.g., TAL proteins, gRNA, etc.).

A number of means of non-chemical stabilization may be used in the practice of the invention. Such means include (a) temperature, (b) pH, (c) ionic strength, (d) complexation with other compounds, (e) the presents of agents that inhibit enzymes that degrade proteins and nucleic acids (e.g., nuclease, inhibitors, protease inhibitors, etc.), and (f) drying.

In some aspects the invention relates to compositions and methods for preparing exsiccated or lyophilized compositions containing gene altering reagents. Reagents that contain only small amounts of solvent (e.g., water) are generally expected to undergo few biological reactions and thus are expected to be relatively stable even at room temperature.

While a number of methods may be used to remove water from samples (e.g., centrifugation in under vacuum or partial vacuum conditions), lyophilization may be carried out according to methods known in the art. In many instances, solvent will be removed by lyophilization. An example of a protocol for lyophilization is the following: (1) Gradient temperature decrease from +20° C. to −40° C. in 5 minutes, (2) −40° C. for 3 hours, (3) gradient temperature increase from −40° C. to −10° C. in 30 minutes, (4) −10° C. for 4 hours, (5) gradient temperature increase from −10° C. to +10° C. in 15 minutes, (6) +10° C. for 2 hours, (7) gradient temperature increase from +10° C. to +30° C. in 15 minutes, and (8) +30° C. for 4-8 hours.

In many instances, glycerol and detergents will not be present in gene altering reagents for certain dry down methods. For example, while glycerol can be present in the lyophilization method referred to above, it is not preferred.

Exsiccation or drying may be employed for stabilizing gene altering reagents. Typically, greater than 80% of gene altering reagents is water. Removal of substantial portions of this water can result in stabilization. Lyophilization, for example, typically lowers the moisture content of a solution to a percentage between 0.3% and 8%. Thus, the invention includes gene altering reagents where the moisture content is from about 0.1% to about 10%, from about 0.5% to about 10%, from about 1% to about 10%, from about 0.1% to about 7%, from about 0.1% to about 5%, from about 0.5% to about 5%, from about 0.5% to about 4%, from about 0.3% to about 6%, from about 0.3% to about 4%, from about 0.3% to about 3%, etc.

One advantage of drying gene altering reagents is that this increases stability at ambient temperature. Thus, in one aspect, the invention provides methods for stabilizing gene altering reagents, as well as compositions generated by such methods.

In some embodiments, cellobiose may be present as a stabilizer at concentrations between 50 mM and 500 mM in a preparation prior to solvent removal. One of the advantage of the use of cellobiose is that it is an effective stabilizer for both lyophilization and preservation of biological molecules (e.g., nucleic acids and proteins), whereas stabilizers of the known art are generally used for either one or the other purpose. Also in many instances, a salt such as KCl or MgCl₂ will be present prior to solvent removal.

A number of means may also be employed for inhibiting the degradation of proteins. One is the presence of one or more protease inhibitors (e.g., phenylmethylsulfonyl fluoride, leupeptin, etc.).

A number of means may also be employed for inhibiting the degradation of nucleic acid molecules, including RNA molecules. One is the presence of one or more RNase inhibitors. A number of commercially available RNase inhibitors are available, including SUPERASE IN™ RNase Inhibitor (cat. no. AM2694), RNASEOUT™ (cat. no. 10777-019), and ANTI-RNase (cat. no. AM2690), all of which are available from Thermo Fisher Scientific.

Capsid proteins from viruses may also be used to stabilize nucleic acid molecules. These viruses may be DNA viruses or RNA viruses. By way of example, when one seeks to stabilize gRNA molecules, one may use capsid proteins from single-stranded RNA viruses such as Coronavirus, SARS virus, Poliovirus, Rhinovirus, and/or Hepatitis A virus.

Further, gRNA may be stabilized by complexation with Cas9 protein. Thus, the invention includes stabilized gene altering reagents containing nucleic acid/protein complexes. Further, such complexes may have solvent removed from them.

### Chemical/Base Stabilization:

Chemical stabilization refers to stabilization means that involve chemical modification of functional components of gene altering reagents (e.g., TAL proteins, gRNA, etc.).

Nucleic acid molecules used in the practice of the invention may be chemically modified. Chemical modification may be employed for a number of purposes. For example, chemical modification may be used to stabilize nucleic acid molecules (e.g., RNA molecules) during storage and/or increase their intracellular half-life. Further, with respect to functional RNA molecules (e.g., gRNA molecules) hairpins may be altered in a manner that stabilizes their structure. This can be done by selection of bases that enhance the formation of hairpin (e.g., G/C content).

Chemical modifications may be of any number of chemical groups and locations. The suitability of a particular chemical modification will vary with the type of RNA molecule and the location within the RNA molecule of the chemical group.

Chemical modifications may be of bases or inter base linkages. Exemplary chemical modifications may include phosphorothioate modifications, 2′-O-methyl modifications, 2′-O-propyl modifications, 2′-O-ethyl modifications, 2′-fluoro modifications, and/or a combination of such modifications. Modified sugars may also be used. Modified sugars include D-ribose, dideoxynucleotides, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH2CH═CH2), 2′-propargyl, 2′-propyl, ethynyl, ethenyl, propenyl, and cyano and the like.

Additional chemical modifications that may be used in the practice of the invention may be found in Hendel et al., Nature Biotech. doi:10.1038/nbt.3290 (2015) and Radhar et al., Proc. Nat'l. Acad. Sci. (USA) doi/10.1073/pnas.1520883112 (2015).

Chemical modifications also include phosphodiester analogs, such as, phosphorothioate, phosphorodithioate, and P ethyoxyphosphodiester, P-ethoxyphosphodiester, P-alkyloxyphosphotriester, methylphosphonate, and nonphosphorus containing linkages (e.g., acetals and amides).

Pseudouridine is the C-glycoside isomer of the uridine and, of the over one hundred different modified nucleosides found in RNA, it is the most prevalent. Pseudouridine is formed by enzymes called pseudouridine synthases, which post-transcriptionally isomerize specific uridine residues in RNA in a process termed pseudouridylation. Pseudouridine is suggested provide protection from radiation. RNA molecules may be stabilized by the addition of pseudouridine and/or 2′-O-methyl modifications at one or more location at or near the 5′ and/or 3′ termini.

Chemical modifications may be increase the storage life and/or intracellular half-life by anywhere from 1.2 to 20 fold (e.g., from about 1.5 to about 20, from about 2 to about 20, from about 1.5 to about 20, from about 1.5 to about 20, from about 1.5 to about 20, from about 1.5 to about 20, from about 1.5 to about 20, from about 1.5 to about 20, etc.).

Chemical modifications may be located at one terminus, both termini and/or interior in nucleic acid molecules. In many instances, chemical modifications will be positioned to inhibit digestion of nucleic acid molecules by exonucleases. In some formats, from one to ten (e.g., from about one to about nine, from about one to about six, from about one to about five, from about one to about four, from about one to about three, from about one to about two, etc.) terminal 5′ and/or 3′ bases will be chemically modified. In more specific formats, the chemical modifications will be either phosphorothioate modifications or 2′-O-methyl modifications or a combination of these modifications.

Chemical modifications may be present in a number from one to twenty (e.g., from about one to about fifteen, from about two to about fifteen, from about three to about fifteen, from about three to about ten, from about three to about eight, from about two to about five, etc.) modifications, such as base modifications, linker modifications and/or sugar modifications.

Many exonucleases are processive in the sense that they remaining attached to their substrates and performing multiple rounds of catalysis before dissociating. Termini of RNA molecules may have different groups present to prevent degradation. As examples, synthetic RNA typically has a 5′ hydroxyl group. RNA produced by in vitro transcription typically has a 5′ triphosphate group. Natural RNA typically has a 5′ monophosphate group. The invention includes stabilized RNA molecules that have one or more of the se 5′ groups, as well as other 5′ groups. As an example, 5′ triphosphate groups may be converted to monophosphate groups by using RNA 5′ pyrophosphohydrolase. Further, 5′ monophosphate groups may be used to improve RNA stability.

A number of additional means may be used to stabilize nucleic acid molecules. For example, a string of polyGs may be added to the 3′ terminus of a nucleic acid molecule to inhibit degradation. In particular, a polyG region may be present in the place of polyA regions found at the 3′ end of mRNA, resulting in increased intracellular half-life on the RNA molecules.

Another way to improve stability of RNA molecules (e.g., gRNA molecules) is to provide these molecules as stabilized loops or hairpins. One example of a modification of such loops is those with contain C/G rich regions. The three hydrogen bonds between these bases in creases loop stability, as compared to loops formed from nucleic acid segments having A/T bases. Stability of RNA molecules can also be increased by the addition of loops, such as tetraloops composed of four pairs of C/G bases. Loops may also be stabilized or introduced as one or both termini. In the case of gRNA, a loop may be introduced at the 5′ terminus. The invention thus includes nucleic acid molecules that contain hairpin regions wherein between 60% to 100% (e.g., from about 65% to about 100%, from about 70% to about 100%, from about 75% to about 100%, from about 80% to about 100%, from about 75% to about 90%, etc.) of the paired bases are C/G pairs. Further, these hairpin regions may contain from about 4 to about 20 paired bases (e.g., from about 5 to about 20 paired bases, from about 6 to about 20 paired bases, from about 7 to about 20 paired bases, from about 5 to about 15 paired bases, from about 6 to about 14 paired bases, etc.).

In some instances, the number of naturally resident hairpins present may be changed to enhance stability of a nucleic acid molecule. The natural tracr molecule forms three hairpins. The final hairpin has 3-5 bases additional at the 3′ end. Tracr molecules, as well as other RNA molecules (e.g., gRNA molecules), may be stabilized by removing some or all of these terminal bases. This is believed to inhibit nuclease initiation. Further, truncation of naturally resident hairpins may result in stabilized RNA molecules by changing solvent exposure.

RNA molecules may also be formed through the introduction of regions that form triplex and/or quadraplex structures, especially at or near the 3′ terminus.

Cross-link groups (e.g., photo-activatable groups) can be added to gRNA (e.g., at or near the 3′ terminus) that allow for cross-linking to the Cas9 protein. This allows for the formation of a stable gRNA/Cas9 complex, where the gRNA is believed to be protected from degradation by the protein.

### Exemplary Gene Altering Reagents:

Three different examples of gene altering systems are zinc finger based systems, TAL effectors based systems, CRISPR based systems (e.g., Cas9 based systems and CPF1 based systems). Each operates by different principles and employ different functional molecules. These systems break down into two groups: (1) Protein based systems (e.g., zinc finger and TAL effectors) and (2) nucleic acid/protein complexed based systems (e.g., CRISPRs).

A. Zinc Finger Based Systems

Zinc-finger nucleases (ZFNs) and meganucleases are examples of genome engineering tools. ZFNs are chimeric proteins consisting of a zinc-finger DNA-binding domain and a nuclease domain. One example of a nuclease domain is the non-specific cleavage domain from the type IIS restriction endonuclease FokI (Kim, Y G; Cha, J., Chandrasegaran, S. Hybrid restriction enzymes: zinc finger fusions to Fok I cleavage domain Proc. Natl. Acad. Sci. USA. 1996 Feb. 6; 93(3):1156-60) typically separated by a linker sequence of 5-7 base pairs. A pair of the FokI cleavage domain is generally required to allow for dimerization of the domain and cleavage of a non-palindromic target sequence from opposite strands. The DNA-binding domains of individual Cys2His2 ZFNs typically contain between 3 and 6 individual zinc-finger repeats and can each recognize between 9 and 18 base pairs.

One problem associated with ZNFs is the possibility of off-target cleavage which may lead to random integration of donor DNA or result in chromosomal rearrangements or even cell death which still raises concern about applicability in higher organisms (Zinc-finger Nuclease-induced Gene Repair With Oligodeoxynucleotides: Wanted and Unwanted Target Locus Modifications Molecular Therapy vol. 18 no. 4, 743-753 (2010)).

B. TAL Effectors Based Systems

Transcription activator-like (TAL) effectors represent a class of DNA binding proteins secreted by plant-pathogenic bacteria of the species, such as Xanthomonas and Ralstonia, via their type III secretion system upon infection of plant cells. Natural TAL effectors specifically have been shown to bind to plant promoter sequences thereby modulating gene expression and activating effector-specific host genes to facilitate bacterial propagation (Römer, P., et al., Plant pathogen recognition mediated by promoter activation of the pepper Bs3 resistance gene. Science 318, 645-648 (2007); Boch, J. & Bonas, U. Xanthomonas AvrBs3 family-type III effectors: discovery and function. Annu. Rev. Phytopathol. 48, 419-436 (2010); Kay, S., et al. U. A bacterial effector acts as a plant transcription factor and induces a cell size regulator. Science 318, 648-651 (2007); Kay, S. & Bonas, U. How Xanthomonas type III effectors manipulate the host plant. Curr. Opin. Microbiol. 12, 37-43 (2009)). Natural TAL effectors are generally characterized by a central repeat domain and a carboxyl-terminal nuclear localization signal sequence (NLS) and a transcriptional activation domain (AD). The central repeat domain typically consists of a variable amount of between 1.5 and 33.5 amino acid repeats that are usually 33-35 residues in length except for a generally shorter carboxyl-terminal repeat referred to as half-repeat. The repeats are mostly identical but differ in certain hypervariable residues. DNA recognition specificity of TAL effectors is mediated by hypervariable residues typically at positions 12 and 13 of each repeat—the so-called repeat variable diresidue (RVD) wherein each RVD targets a specific nucleotide in a given DNA sequence. Thus, the sequential order of repeats in a TAL protein tends to correlate with a defined linear order of nucleotides in a given DNA sequence. The underlying RVD code of some naturally occurring TAL effectors has been identified, allowing prediction of the sequential repeat order required to bind to a given DNA sequence (Boch, J. et al. Breaking the code of DNA binding specificity of TAL-type III effectors. Science 326, 1509-1512 (2009); Moscou, M. J. & Bogdanove, A. J. A simple cipher governs DNA recognition by TAL effectors. Science 326, 1501 (2009)). Further, TAL effectors generated with new repeat combinations have been shown to bind to target sequences predicted by this code. It has been shown that the target DNA sequence generally start with a 5′ thymine base to be recognized by the TAL protein.

The modular structure of TALs allows for combination of the DNA binding domain with effector molecules such as nucleases. In particular, TAL effector nucleases allow for the development of new genome engineering tools known.

C. CRISPR Based Systems

Gene altering reagents may be based upon CRISPR systems. The term “CRISPR” is a general term that applies to three types of systems, and system sub-types. In general, the term CRISPR refers to the repetitive regions that encode CRISPR system components (e.g., encoded crRNAs). Three types of CRISPR systems (see Table 1) have been identified, each with differing features.

TABLE 1
CRISPR System Types Overview

| System | Features | Examples |
|---|---|---|
| Type I | Multiple proteins (5-7 proteins typical), | Staphylococcus |
| | crRNA, requires PAM. DNA Cleavage | epidermidis (Type IA) |
| | is catalyzed by Cas3. | |
| Type II | 3-4 proteins (one protein (Cas9) has | Streptococcus |
| | nuclease activity) two RNAs, requires | pyogenes CRISPR/ |
| | PAMs. Target DNA cleavage catalyzed | Cas9, Francisella |
| | by Cas9 and RNA components. | novicida U112 Cpf1 |
| Type III | Five or six proteins required for cutting, | S. epidermidis |
| | number of required RNAs unknown but | (Type IIIA); |
| | expected to be 1, PAMs not required. | P. furiosus |
| | Type IIIB systems have the ability to | (Type IIIB). |
| | target RNA. | |

While the invention has numerous aspects and variations associated with it, the Type II CRISPR/Cas9 system has been chosen as a point of reference for explanation herein.

In certain aspects, the invention provides stabilized crRNAs, tracrRNAs, and/or guide RNAs (gRNAs), as well as collections of such RNA molecules.

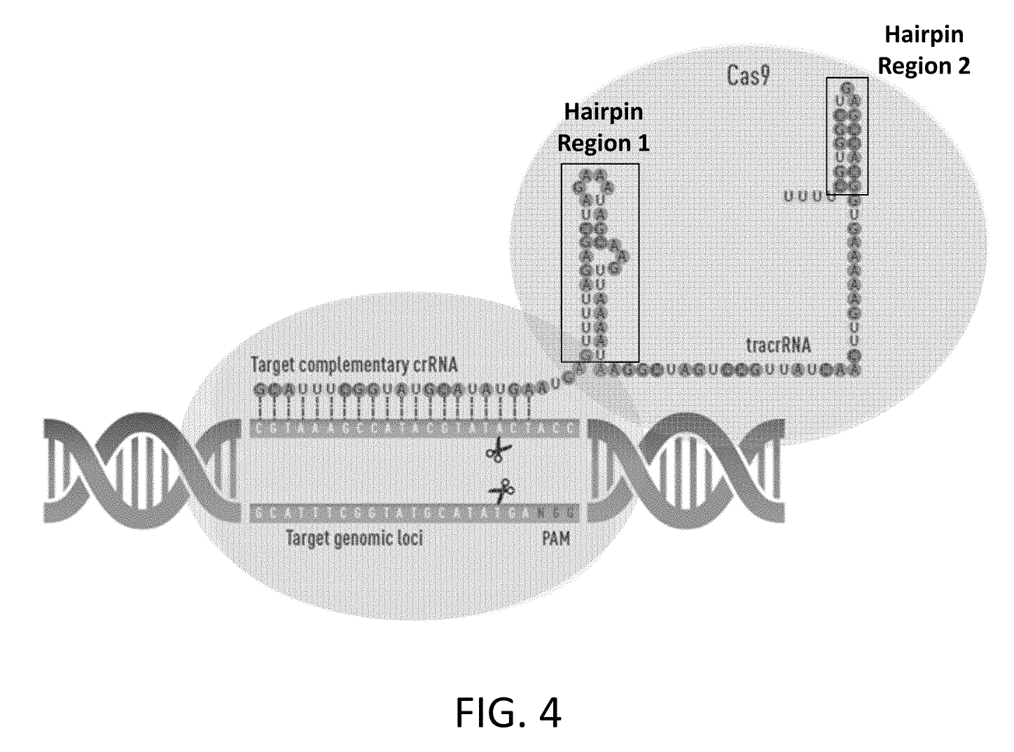

FIG. 4 shows components and molecular interactions associated with a Type II CRISPR system. In this instance, the Cas9 mediated Streptococcus pyogenes system is exemplified. A gRNA is shown in FIG. 4 hybridizing to both target DNA (Hybridization Region 1) and tracrRNA (Hybridization Region 2). In this system, these two RNA molecules serve to bring the Cas9 protein to the target DNA sequence is a manner that allows for cutting of the target DNA. The target DNA is cut at two sites, to form a double-stranded break.

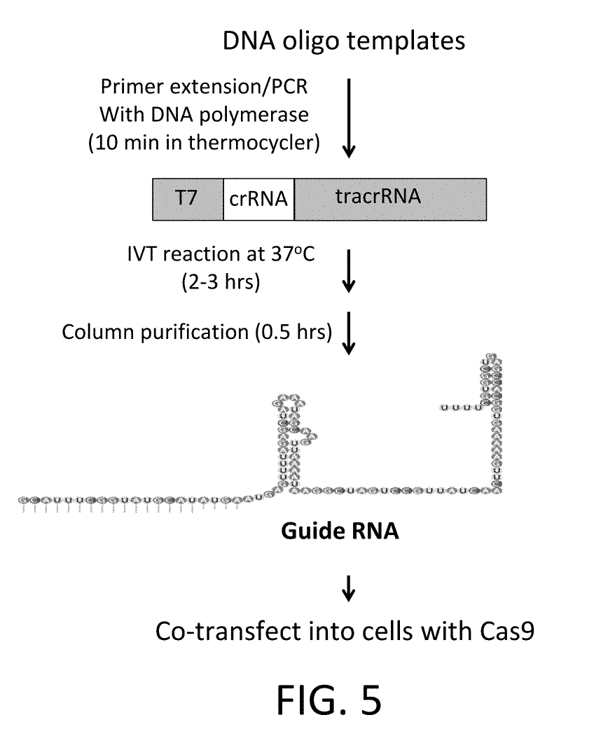

FIG. 5 shows an exemplary workflow of the invention. The schematic in FIG. 5 shows oligonucleotides designed to generate a DNA molecule where the guide RNA coding region is operably linked to a T7 promoter. In this work flow DNA oligonucleotides either alone or in conjunction with double-stranded DNA are used to generate, via PCR, a DNA molecule encoding a guide RNA operably linked to a promoter suitable for in vitro transcription. The DNA molecule is then transcribed in vitro to generate guide RNA. The guide RNA may then be “cleaned up” by, for example, column purification or bead based methods. The guide RNA is then suitable for use by, as examples, (1) direct introduction into a cell or (2) introduction into a cell after being complexed with one or more CRISPR protein. Nucleic acid operably connected to a T7 promoter can be transcribed in mammalian cells when these cells contain T7 RNA polymerase (Lieber et al., Nucleic Acids Res., 17: 8485-8493 (1989)). Of course, other promoters functional in eukaryotic cells (e.g., CMV promoter, U6 promoter, H1 promoter, etc.) could also be used for the intracellular production of guide RNA. The H1 promoter, for example, is about 300 base pairs in length. One advantage of the T7 promoter is its small size (20 base pairs).

One advantage of using chemically synthesized and in vitro transcribed RNA is that chemically modified bases may be introduced into the RNA molecules.

Dried or lyophilized gene altering complexes may also be used. A number of formulations may be used for dried or lyophilized gene altering reagents that have been allowed to form complexes. In many instances, complexes may be formed using CRISPR system reagents.

Dried or lyophilized gene altering reagents complexes may be tested and/or used by the introduction of such complexes in cells (e.g., U2OS cells, HEK293 cells, etc.). Further, complexes may be prepared in or placed into in multi-well formats in 1× to 5× amounts. For Cas9 mRNA formats, LIPOFECTAMINE® RNAiMAX, or equivalent, may be used. For Cas9 protein formats CRISPRMAX, or equivalent, may be used for lipid nanoparticle based transfection.

Cas9/gRNA are exemplary conditions are used below for purposes of illustration.

Format 1:

No transfection reagent or Cas9. 1 to 5 μg of gRNA is added to wells of multiwell plates. The plate and contents is vacuum dried, then stored at different temperatures. Prior to use gRNA is resuspended to an appropriate concentration. Cas9 expressing stable cells or cells co-transfect with Cas9 and a suitable transfection reagent are added to the wells. In some aspects, one or more (e.g., two) TAL protein, TAL mRNA encoding one or more (e.g., two) TAL protein or DNA encoding one or more (e.g., two) TAL protein may be added to the wells instead in conjunction with or instead of gRNA.

Format 2:

20 ng of IVT generated gRNA (20 ng/μl) and 10Ong Cas9mRNA (100 ng/μl) are mixed to form complexes and added to wells of multiwell plates. The plate and contents is vacuum dried, then stored at different temperatures. Prior to transfect, the samples are resuspended in RNAse and DNAse free water or OPTI-MEM™ culture medium. Following resuspension of the dried samples, LIPORFECTAMINE® RNAiMAX/OPTI-MEM™ mix (prepared using 0.6 μl of LIPOFECTAMINE® RNAiMAX and 4.40 OPTI-MEM™ per well) is added to the gRNA-Cas9 complexes and then applied to 15,000-20,000 cells/well. In some aspects, one or more (e.g., two) TAL protein, TAL mRNA encoding one or more (e.g., two) TAL protein or DNA encoding one or more (e.g., two) TAL protein may be added to the wells instead in conjunction with or instead of gRNA.

Format 3:

IVT gRNA (20 ng/well at 20 ng/μl) and Cas9 mRNA (10Ong/well at 100 ng/μl) is precomplexed with LIPOFECTAMINE® RNAiMAX (0.6 μl/well) and vacuum dry. Dried pre-complexed samples are resuspended in OPTI-MEM™ and used for transfection. In some aspects, one or more (e.g., two) TAL protein, TAL mRNA encoding one or more (e.g., two) TAL protein or DNA encoding one or more (e.g., two) TAL protein may be added to the wells instead in conjunction with or instead of gRNA.

Format 4:

IVT generated gRNA (20 ng/well) and Cas9 mRNA (10Ong/well) is precomplexed with LIPOFECTAMINE® RNAiMAX (or equivalent) and OPTI-MEM™ (4.40 per well). The mixture is vacuum dried. Prior to use samples are resuspended in OPTI-MEM™ and/or water and applied to cells in 96 well format. In some aspects, one or more (e.g., two) TAL protein, TAL mRNA encoding one or more (e.g., two) TAL protein or DNA encoding one or more (e.g., two) TAL protein may be added to the wells instead in conjunction with or instead of gRNA.

Format 5:

IVT generated gRNA is precomplexed with LIPOFECTAMINE® RNAiMAX or LIPOFECTAMINE® MESSENGERMAX™ (with or without OPTI-MEM™). This format may be used with stable Cas9 expressing cell lines. Amounts of components used are the same or similar to above described formats. In some aspects, one or more (e.g., two) TAL protein, TAL mRNA encoding one or more (e.g., two) TAL protein or DNA encoding one or more (e.g., two) TAL protein may be added to the wells instead in conjunction with or instead of gRNA.

Format 6:

Formats 1-4 with donor DNA (e.g., single-stranded DNA). In some aspects, one or more (e.g., two) TAL protein, TAL mRNA encoding one or more (e.g., two) TAL protein or DNA encoding one or more (e.g., two) TAL protein may be added to the wells instead in conjunction with or instead of gRNA.

### Gene Alteration Activities:

Reagents of the invention can have any number of activities. For example, the reagents may comprise fusion proteins that have one or more heterologous domains (e.g., one, two, three, four, five, etc.). Fusion proteins may comprise any additional protein sequence, and optionally a linker sequence between any two domains. Examples of protein domains that may be a fusion protein component include, without limitation, epitope tags, reporter gene sequences, and one or more domain having one or more of the following activities: methylase activity, demethylase activity, transcription activation activity, transcription repression activity, transcription release factor activity, histone modification activity (e.g., acetylation activity, deacetylation activity, phosphorylation activity, dephosphorylation activity, methylation activity, demethylation activity, etc.) RNA cleavage activity, and nucleic acid binding activity.

In particular, provided herein, in part, are gene altering reagents, which comprise at least one nuclear localization signal, at least one domain with a functional activity (e.g., nuclease, methylase, etc.), and at least one domain that interacts with a target locus or at least one domain that interaction with a nucleic acid molecule that interacts with a target locus.

Gene altering reagents may be employed to activate or repress transcription. For example, “dead” Cas9 (i.e., dCas9) proteins without nuclease activity may be used for non-code altering purposes. dCas9-transcriptional activator fusion protein (e.g., dCas9-VP64) may be used in conjunction with a guide RNA to activate transcription of nucleic acid associated with a target locus. Similarly, dCas9-repressor fusions (e.g., dCas9-KRAB transcriptional repressor) may be used to repress transcription of nucleic acid associated with a target locus. Transcriptional activation and repression such as the referred to above are discussed in, for example, Kearns et al., Cas9 effector-mediated regulation of transcription and differentiation in human pluripotent stem cells, Development, 141:219-223 (2014).

The invention thus includes compositions and methods for the production and use of gene altering reagents for the activation and repression of transcription.

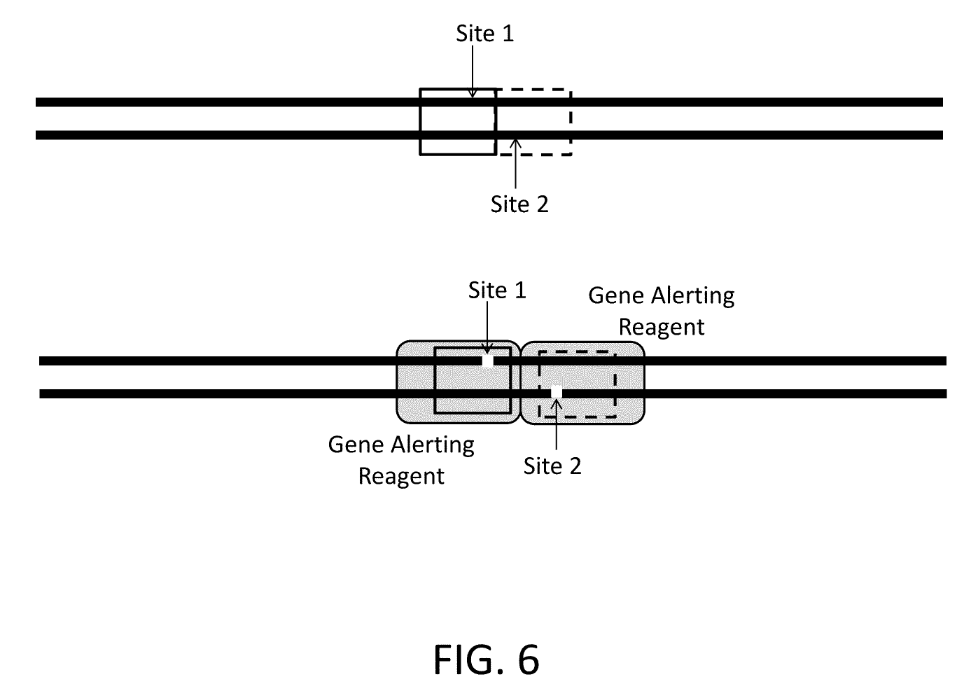

FIG. 6 shows the selection of two closely associated sites that form a target locus. Each of the sites (Site 1 and Site 2) binds a gene altering reagent with nicking activity. One purpose of this is to minimize “off target” cutting of nucleic acid.

The two sites exemplified in FIG. 6 will generally be located sufficiently close to each other so that the double-stranded nucleic acid containing the nick breaks. While this distance will vary with factors such as the AT/CG content of the region, the nick sites will generally be within 200 base pairs of each other (e.g., from about 1 to about 200, from about 10 to about 200, from about 25 to about 200, from about 40 to about 200, from about 50 to about 200, from about 60 to about 200, from about 1 to about 100, from about 10 to about 100, from about 20 to about 100, from about 30 to about 100, from about 40 to about 100, from about 50 to about 100, from about 1 to about 60, from about 10 to about 60, from about 20 to about 60, from about 30 to about 60, from about 40 to about 60, from about 1 to about 35, from about 5 to about 35, from about 10 to about 35, from about 20 to about 35, from about 25 to about 35, from about 1 to about 25, from about 10 to about 25, from about 15 to about 25, from about 2 to about 15, from about 5 to about 15, etc. base pairs).

The nicking activity may be accomplished in a number of ways. For example, when the gene altering reagent is Cas9, the Cas9 protein has two domains, termed RuvC and HNH, that nick different strands of double-stranded nucleic acid. Cas9 proteins may be altered to inactivate one domain or the other. The result is that two Cas9 proteins are required to nick the target locus in order for a double—stranded break to occur. For example, an aspartate-to-alanine substitution (D10A) in the RuvC catalytic domain of Cas9 from S. pyogenes converts Cas9 from a nuclease that cleaves both strands to a nickase (cleaves a single strand). Other examples of mutations that render Cas9 a nickase include H840A, N854A, and N863A.

CRISPR proteins (e.g., Cas9) with nickase activities may be used in combination with guide sequences (e.g., two guide sequences) which target respectively sense and antisense strands of the DNA target.

Another way to generate double-stranded breaks in nucleic acid using nickase activity is by using CRISPR proteins that lack nuclease activity linked to a heterologous nuclease domain. One example of this is a mutated form of Cas9, referred to as dCas9, linked to FokI domain. FokI domains require dimerization for nuclease activity. Thus, in such instances, CRISPR RNA molecules are used to bring two dCas9-FokI fusion proteins into sufficiently close proximity to generate nuclease activity that results in the formation of a double-stranded cut. Methods of this type are set out in Tsai et al., “Dimeric CRISPR RNA-guided FokI nucleases for highly specific genome editing,” Nature Biotech., 32:569-576 (2014) and Guilinger et al., “Fusion of catalytically inactive Cas9 to FokI nuclease improves the specificity of genome modification,” Nature Biotech., 32:577-582 (2014).

Another way to minimize “off target” cutting of nucleic acid is through the use of nucleases that are inactive until they dimerize. One example of such a nuclease is FokI. Zinc finger proteins and TAL effector proteins have been designed to bind different sites on a nucleic acid molecule to allow for the FokI domains to dimerize, resulting in reconstitution of nuclease activity.

The invention thus includes gene altering reagents that recognize more than one locus on a nucleic acid molecule. In many instances, the distance between the recognition sites will be in the same range as the nick sites referred to in reference to FIG. 6.

Functional activities can be measured in any number of ways. For example, activities based upon induction or repression of expression can be measured by assessing increases or decreases in transcription and/or translation.

When functional activities related to the cleavage of DNA (e.g., intracellular DNA), then a number of commercial products are available for the detection of nucleic acid cleavage. One such product is the GENEART® Genomic Cleavage Detection Kit (cat. no. A24372), available from Thermo Fisher Scientific. Additional assay may be found in U.S. patent application Ser. No. 14/879,872, filed Oct. 9, 2015, entitled “CRISPR Oligonucleotides and Gene Editing, the entire disclosure of which is incorporated herein by reference.

### Reagent Mixtures and Formats:

A number of compounds that do not have direct gene alteration activity may be included in the reagent mixture. One such set of compounds is transfection reagents. These may be included to for minimal addition to the gene altering reagent as part of an experimental protocol.

Transfection agents suitable for use with the invention include transfection agents that facilitate the introduction of RNA, DNA and proteins into cells. Exemplary transfection reagents include TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293FECTIN™, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), LIPOFECTAMINE™ CRISPRMAX™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFx-10TH (Promega), TFx-20TH (Promega), TFx-50TH (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.).

Gene altering reagents may be set up in a format such that minimal additions are required for gene altering activity. In one exemplary format, donor nucleic acid, a pair of ZNF-FokI fusion proteins, and a transfection reagent are lyophilized in a well of a 96 well plate. Cells in a culture medium are added to the well with the lyophilized gene altering reagent and another well that does not contain the gene altering reagent (a control well). The efficiency of homologous recombination at the target locus is later measured for both samples.

In some instances, the gene altering reagent will contain gRNA and Cas9 protein will be expressed by cells contacted with the gRNA. gRNA taken up by the cells will then associate with Cas9 protein expressed intracellularly to reconstitute gene altering activities. Where appropriate, these cells may be contacted with donor nucleic acid prior to, simultaneously with, or after the cells have been contacted with gRNA.

The invention further includes collections of gene altering reagents with specificity for individual target sites. For example, the invention includes collections of gene altering reagents with specificity for target sites within particular types of cell (e.g., human cells). The members of such collection of cells may be generated based upon sequence information for these particular types of cells. As an example, one such collection could be generated using the complete genome sequence of a particular type of cell. The genome sequence data can be used to generate a library of gene altering reagents with specificity for the coding region of each gene within the human genome.

Collections or libraries of crRNA molecules or the invention may include a wide variety of individual molecules such as from about five to about 100,000 (e.g., from about 50 to about 100,000, from about 200 to about 100,000, from about 500 to about 100,000, from about 800 to about 100,000, from about 1,000 to about 100,000, from about 2,000 to about 100,000, from about 4,000 to about 100,000, from about 5,000 to about 100,000, from about 50 to about 50,000, from about 100 to about 50,000, from about 500 to about 50,000, from about 1,000 to about 50,000, from about 2,000 to about 50,000, from about 4,000 to about 50,000, from about 50 to about 10,000, from about 100 to about 10,000, from about 200 to about 10,000, from about 500 to about 10,000, from about 1,000 to about 10,000, from about 2,000 to about 10,000, from about 4,000 to about 10,000, from about 50 to about 5,000, from about 100 to about 5,000, from about 500 to about 5,000, from about 1,000 to about 5,000, from about 50 to about 2,000, from about 100 to about 2,000, from about 500 to about 2,000, etc.).

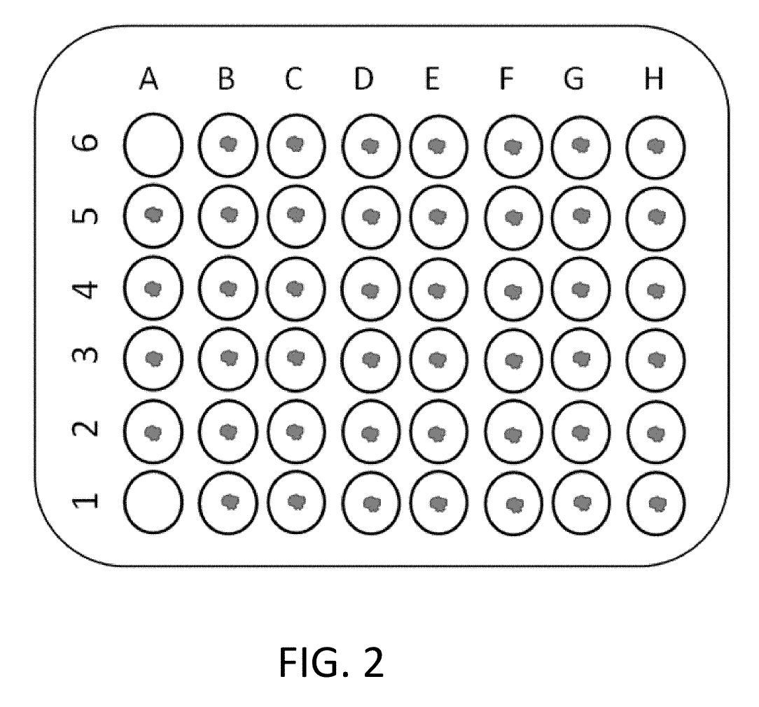

Gene altering reagents used in the practice of the invention may be stored in a number of different formats. For example, RNA molecules may be stored in tubes (e.g., 1.5 ml microcentrifuge tubes) or in the wells of plates (e.g., 96 well, 384 well, or 1536 well plates). One exemplary format is shown in FIG. 2. In this figure, wells A,1 and A,6 are control wells and contain no gene altering reagents. The other wells contain desiccated gene altering reagents that may be reconstituted with, for example, culture media containing cells. Further, each well may contain a gene altering reagent with binding specificity for a different target locus.

### Vector Components and Cells:

A number of functional nucleic acid components (e.g., promoters, polyA signal, origins of replication, selectable markers, etc.) may be used in the practice of the invention. The choice of functional nucleic acid components used in the practice of the invention, when employed, will vary greatly with the nature of the use and the specifics of the system (e.g., intracellular, extracellular, in vitro transcription, coupled in vitro transcription/translation, etc.).

Promoter choice depends upon a number of factors such as the expression products and the type of cell or system that is used. For example, non-mRNA molecules are often produced using RNA polymerase I or III promoters. mRNA is generally transcribed using RNA polymerase II promoters. There are exceptions, however. One is microRNA expression systems where a microRNA can be transcribed from DNA using an RNA polymerase II promoter (e.g., the CMV promoter). While RNA polymerase II promoters do not have “sharp” stop and start points, microRNAs tend to be processed by removal of 5′ and 3′ termini. Thus, “extra” RNA segments at the termini are removed. mRNA (e.g., cas9 mRNA) is normally produced via RNA polymerase II promoters.

The choice of a specific promoter varies with the particular application. For example, the T7, T3 and SP6 promoters are often used for in vitro transcription and in vitro transcription/translations systems. When intracellular expression in desired, the promoter or promoters used will generally be designed to function efficiently within the cells employed. The CMV promoter, for example, is a strong promoter for use within mammalian cells. The hybrid Hsp70A-Rbc S2 promoter is a constitutive promoter that functions well in eukaryotic algae such as Chlamydomonas reinhardtii. (see the product manual “GeneArt® Chlamydomonas Protein Expression Kit”, cat. no. A24244, version B.0, from Life Technologies Corp., Carlsbad, Calif.). Additional promoters that may be used in the practice of the invention include AOX1, GAP, cauliflower mosaic virus 35S, pGC1, EF1α, and Hsp70 promoters.

The DNA segment in the expression vector is operatively linked to an appropriate expression control sequence(s) (promoter) to direct RNA synthesis. Suitable eukaryotic promoters include the CMV immediate early promoter, the HSV thymidine kinase promoter, the early and late SV40 promoters, the promoters of retroviral LTRs, such as those of the Rous Sarcoma Virus (RSV), and metallothionein promoters, such as the mouse metallothionein-I promoter. Exemplary promoters suitable for use with the invention are from the type III class of RNA polymerase III promoters. Additionally, the promoters may be selected from the group consisting of the U6 and H1 promoters. The U6 and H1 promoters are both members of the type III class of RNA polymerase III promoters.

RNA polymerase III promoters are suitable for in vivo transcription of nucleic acid molecules produced by methods of the invention. For example, linear DNA molecules produced as set out in FIG. 5 may be introduced into cells and transcribed by, for example, naturally resident intracellular transcriptional processes.

Promoters in compositions and methods of the invention may also be inducible, in that expression may be turned “on” or “off.” For example, a tetracycline-regulatable system employing the U6 promoter may be used to control the production of siRNA. Expression vectors may or may not contain a ribosome binding site for translation initiation and a transcription terminator. Vectors may also include appropriate sequences for amplifying expression.

Cells suitable for use with the present invention include a wide variety of prokaryotic and eukaryotic cells. In many instances, one or more CRISPR system components will not be naturally associated with the cell (i.e., will be exogenous to the cell).

Representative cells that may be used in the practice of the invention include, but are not limited to, bacterial cells, yeast cells, plant cells and animal cells. Exemplary bacterial cells include Escherichia spp. cells (particularly E. coli cells and most particularly E. coli strains DH10B, Stb12, DH5α, DB3, DB3.1), Bacillus spp. cells (particularly B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells). Exemplary animal cells include insect cells (most particularly Drosophila melanogaster cells, Spodoptera frugiperda Sf9 and Sf21 cells and Trichoplusa High-Five cells), nematode cells (particularly C. elegans cells), avian cells, amphibian cells (particularly Xenopus laevis cells), reptilian cells, and mammalian cells (more particularly NIH3T3, CHO, COS, VERO, BHK CHO-K1, BHK-21, HeLa, COS-7, HEK 293, HEK 293T, HT1080, PC12, MDCK, C2C12, Jurkat, NIH3T3, K-562, TF-1, P19 and human embryonic stem cells like clone H9 (Wicell, Madison, Wis., USA)). Exemplary yeast cells include Saccharomyces cerevisiae cells and Pichia pastoris cells. These and other cells are available commercially, for example, from Thermo-Fisher Scientific (Waltham, Mass.), the American Type Culture Collection, and Agricultural Research Culture Collection (NRRL; Peoria, Ill.). Exemplary plant cells include cells such as those derived from barley, wheat, rice, soybean, potato, arabidopsis and tobacco (e.g., Nicotiana tabacum SR1).

### EXAMPLES

### Example 1: Preparation of Dried Reagents

Spray Drying: A dry formulation of guide RNA is prepared from guide RNA, 1,2-Dipalmitoyl-sn-Glycero-3-Phosphocholine (DPPC), sucrose, and albumin (20:40:20:20 by weight). An aqueous solution containing 15 mg of siRNA, 15 mg of albumin, and 15 mg of sucrose (total volume 7.5 ml) is mixed with 17.5 ml of ethanol containing 30 mg of DPPC. Prior to combining the solutions they are mixed with a magnetic stir bar. After the aqueous solution is added to the organic solution, the combined solution was mixed by magnetic stir bar, at room temperature for about 6 minutes before the solution is spray dried. Conditions for spray drying are Tᵢₙₗₑₜ=95° C., Tₒᵤₜₗₑₜ=55° C., atomization/drying gas flow rate is 600 L/hr.

### Example 2: Preparation of Dried Reagents

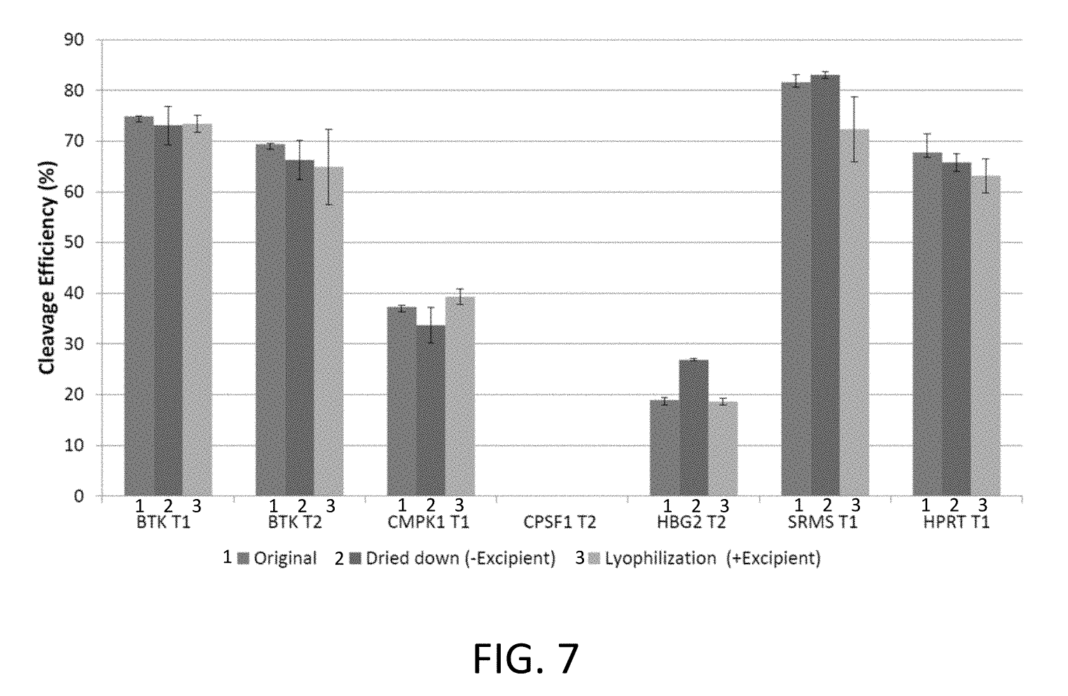

Twenty μl of in vitro transcribed guide RNA stock (0.5 μg to 1 μg per well) in RNA storage buffer (1 mM sodium citrate pH 6.4) were lyophilized in 96 well plate format and maintained at −20 C. Prior to transfection into U2OS Cas9 stable cells, the dried down gRNA were centrifuged briefly and resuspended in 20 μl DNAse/RNAse free water. RNA concentration was measured prior to transfection using QUANT-IT™ RNA BR Assay Kit. One day prior to transfection, 10,000 cells were seeded per well in a 96 well plate format. On the day of transfection for each well, except the untransfected controls, 20 ng of gRNA was added to 5 μl of OPTI-MEM® medium, followed by addition of 5 μl of Opti-MEM containing 1.5 μl of LIPOFECTAMINE™ RNAiMAX. The resulting transfection mix was incubated at room temperature for 10 minutes and then added to the cells. The plate containing transfected cells was incubated at 37° C. for 48 hours in a 5% CO2 incubator. The percentage of locus-specific indel formation was measured by GENEART® Genomic Cleavage Detection Kit (Thermo Fisher Scientific, cat. no. A24372). The band intensities were quantitated using built-in software in Alpha Imager (Bio-Rad). FIG. 7 shows cleavage efficiency results obtained for six different genes. In case of BTK gene, two different genomic loci were tested. For each sample tested, dried down samples with either excipient or no excipient were compared to a non-lyophilized IVT gRNA in RNA storage buffer.

While the foregoing embodiments have been described in some detail for purposes of clarity and understanding, it will be clear to one skilled in the art from a reading of this disclosure that various changes in form and detail can be made without departing from the true scope of the embodiments disclosed herein. For example, all the techniques, apparatuses, systems and methods described above can be used in various combinations.

Exemplary Subject Matter of the Invention is Represented by the Following Clauses:

Clause 1. A method for preparing one or more stabilized gene altering reagent, the method comprising:

(a) preparing one or more gene altering reagent in a solvent, and

(b) removing more than 80% of the solvent of (a).

Clause 2. The method of clause 1, wherein the solvent is water, one or more alcohol or a mixture of water and one or more alcohol.

Clause 3. The method of any one of the previous clauses, wherein at least one of the one or more gene altering reagents is one or more reagent selected from the group consisting of:

(a) a TAL effector-nuclease fusion protein,

(b) a nucleic acid molecule encoding a TAL effector-nuclease fusion protein,

(c) a zinc finger-nuclease fusion protein,

(d) a nucleic acid molecule encoding a zinc finger-nuclease fusion protein,

(e) a Cas9 protein,

(f) a nucleic acid molecule encoding a Cas9 protein,

(g) a guide RNA, and

(h) a nucleic acid molecule encoding a guide RNA.

Clause 4. The method of clause 2, wherein the water is removed by lyophilization, spray drying, spray freeze drying, supercritical fluid drying, or vacuum centrifugation.

Clause 5. The method of any one of the previous clauses, wherein is between 80% and 99.5% of the solvent removed from the one or more gene altering reagents in aqueous solution.

Clause 6. The method any one of the previous clauses, wherein individual gene altering reagents are placed in two or more wells of a multiwell plate.

Clause 7. The method of clause 6, wherein the individual gene altering reagents are added to wells of the multiwell plate in the solvent.

Clause 8. The method according to clauses 6 through 7, wherein some or all of the aqueous solvent is removed from the individual gene altering reagents while the individual gene altering reagents are in wells of the multiwall plate.

Clause 9. The method of clause 6, wherein between 50 and 100 individual gene altering reagents are placed in different wells of the multiwell plate.

Clause 10. The method according to clauses 6 through 9, wherein the individual gene altering reagents bind to different nucleotide sequences of the genome of the same organism.

Clause 11. The method of any one of the previous clauses, wherein the aqueous solution contains one or more component selected from the group consisting of:

(a) one or more buffer,

(b) one or more protease inhibitor,

(c) one or more nuclease inhibitor,

(d) one or more salt,

(e) one or more carbohydrate,

(f) one or more transfection reagent,

(g) one or more polyamine, and

(h) one or more culture medium.

Clause 12. The method of clause 11, wherein the carbohydrate is one or more of the following: sucrose, trehalose, lactosucrose, or a cyclodextrin.

Clause 13. The method of any one of the previous clauses, wherein the pH of the aqueous solution prior to the removal of the water is between 4 to about 11.

Clause 14. A method for storing one or more gene altering reagents, the method comprising:
(a) preparing one or more gene altering reagents in aqueous solution,
(b) removing more than 90% of the water from the aqueous solution prepared in (a), and
(c) placing one or more gene altering reagents under conditions where greater than 75% of gene altering functional activity is retained after 30 days of storage.

Clause 15. The method of clause 14, wherein greater than 90% of gene altering functional activity of at least one or the one or more gene altering reagents is retained after at least 30 days of storage.

Clause 16. The method of clauses 14 or 15, wherein greater than 90% of gene altering functional activity of at least one or the one or more gene altering reagents is retained after 120 days of storage.

Clause 17. The method of clauses 14, 15 or 16, wherein more than one of the one or more gene altering reagents are stored in the same storage container.

Clause 18. The method of clause 17, wherein the storage container is a multiwell plate.

Clause 19. The method of clause 17, wherein the individual gene altering reagents bind to different nucleotide sequences of the genome of the same organism.

Clause 20. The method of clauses 14, 15, 16, 17, 18, or 19, wherein the one or more gene altering reagents are stored at −20° C., 4° C., or between 20° C. and 30° C.

Clause 21. A composition comprising one or more stabilized gene altering reagents, the composition comprising one or more gene altering reagent, wherein the moisture content of the gene altering reagent is less than 10% (w/w).

Clause 22. The composition of clause 21, wherein the moisture content is from about 0.2% to about 8%.

Clause 23. The composition of clauses 21 or 22, wherein at least one of the one or more gene altering reagents is one or more reagent selected from the groups consisting of:

(a) a TAL effector-nuclease fusion protein,

(b) a nucleic acid molecule encoding a TAL effector-nuclease fusion protein,

(c) a zinc finger-nuclease fusion protein,

(d) a nucleic acid molecule encoding a zinc finger-nuclease fusion protein,

(e) a Cas9 protein,

(f) a nucleic acid molecule encoding a Cas9 protein,

(g) a guide RNA, and

(h) a nucleic acid molecule encoding a guide RNA.

Clause 24. The composition of clauses 21, 22, or 23, wherein the stabilized reagent contains one or more component selected from the group consisting of:

(a) one or more buffer,

(b) one or more protease inhibitor,

(c) one or more nuclease inhibitor,

(d) one or more salt,

(e) one or more carbohydrate,

(f) one or more transfection reagent,

(g) one or more polyamine, and

(h) one or more culture medium.

Clause 25. The composition of clauses 21, 22, 23, or 24, wherein between 50 and 100 individual stabilized gene altering reagents are placed in different wells of a multiwell plate.

1.-25. (canceled)

26. A composition comprising at least one CRISPR complex,
wherein the moisture content of the CRISPR complex is less than 10% (w/w), and
wherein the CRISPR complex retains at least 75% of its functional activity for at least 30 days.

27. The composition of claim 26, wherein the CRISPR complex comprises a Cas9 protein.

28. The composition of claim 26, wherein the CRISPR complex comprises a Cpf1 protein.

29. The composition of claim 26, wherein the CRISPR complex comprises a guide RNA.

30. The composition of claim 26, wherein the CRISPR complex comprises a tracrRNA and a crRNA.

31. The composition of claim 26, further comprising a transfection reagent.

32. The composition of claim 26, further comprising one or more donor nucleic acid molecules.

33. The composition of claim 26, wherein the composition is provided in an array format.

34. The composition of claim 26, wherein the composition retains at least 75% of its functional activity when stored at room temperature for at least 30 days.

35. The composition of claim 26, wherein the composition retains at least 75% of its functional activity when stored at −20° C. for at least 30 days.

36. The composition of claim 26, wherein the composition retains at least 75% of its functional activity when stored at −70° C. for at least 30 days.

37. The composition of claim 26, further comprising a stabilizing agent.

38. The composition of claim 37, wherein the stabilization agent is selected from the group consisting of: one or more buffers, one or more protease inhibitors, one or more nuclease inhibitors, one or more salts, one or more carbohydrates, one or more polyamines, one or more culture media, and any combination thereof.

39. The composition of claim 37, wherein the composition comprises a polyamine.

40. The composition of claim 26, wherein the CRISPR complex retains at least 80% of its functional activity for at least 30 days.

41. The composition of claim 26, wherein the CRISPR complex retains at least 90% of its functional activity for at least 30 days.

42. The composition of claim 26, wherein the at least one CRISPR complex is lyophilized.

43. The composition of claim 26, wherein the CRISPR complex comprises a ribonucleic acid, the ribonucleic acid comprising a chemical modification.

44. The composition of claim 26, comprising a library of CRISPR complexes.

45. The composition of claim 26, wherein the CRISPR complex retains at least 75% of its functional activity for at least 90 days.
